(19) United States
(12) Patent Application Publication
(10) Pub. No.: US 2024/0277882 A1
(43) Pub. Date: Aug. 22, 2024

(54) Smart Scent Container Tag Tracking

(71) Applicant: Pura Scents, Inc., Pleasant Grove, UT (US)

(72) Inventors: Juan Carlos Altuna, South Jordan, UT (US); Christopher Michael Cooper, Saratoga Springs, UT (US); Celeste Chubak, West Valley City, UT (US); David Freitag, Pleasant Grove, UT (US); Jeyaganesh Subramaniaraja, Pleasant Grove, UT (US); Caleb Probst, Nephi, UT (US); John Adam Purin, Ogden, UT (US); Jacob Adam Hougaard, Orem, UT (US); Cody Hardcastle, Lehi, UT (US); Trevor Davis, American Fork, UT (US); Zachary Dennis Blume, Orem, UT (US); Andrew Wallin, Millcreek, UT (US)

(21) Appl. No.: 18/443,943

(22) Filed: Feb. 16, 2024

Related U.S. Application Data
(60) Provisional application No. 63/485,869, filed on Feb. 17, 2023.

(51) Int. Cl. A61L 9/03

(52) U.S. Cl. CPC A61L 9/03; A61L 2209/111; A61L 2209/133

(57) ABSTRACT

A scent dispensing apparatus may include a scent container retaining apparatus configured to receive and securely retain a scent container, wherein the scent container includes a smart vial tag, a smart vial sensor directionally positioned to have a direct line of sight with the smart vial tag to read the smart vial tag when the scent container is securely retained in the scent retaining apparatus, and a fragrance management application that receives an identity of the smart vial tag from the smart vial sensor and generates a unique container profile for the scent container.

### BACKGROUND

The present disclosure relates to tracking of smart container tags on scent containers in a scent dispenser used for scent dispensation.

Existing solutions for dispensing scents within a location, such as a home, include conventional scented candles, incense stocks, hanging air fresheners, and electric fragrance diffusers, such as those that plug into an outlet socket and include a heating element to heat a fragrant substance. In some cases, a porous substrate is saturated with the fragrant substance and then placed adjacent to a heat source, which heats up the fragrant substance to diffuse it within a predetermined space.

While functional, these solutions are limited. For example, it is often difficult to tell when a fragrance dispenser has run out of fragrance, as the dispenser typically lacks any sort of indicator and one is left to smell the dispenser manually to determine how much fragrance is actually left, which is ineffective as the dispenser itself may still smell like the diffuse fragrance up close that may have little effect in the area that is to be perfumed. In addition, if the users wishes to change a fragrance, the user has to manually open the dispenser to replace the cartridge in the dispenser, or in the case of hanging dispensers, replace it with a fresh version and the scent dispenser has no way of knowing what types of cartridges or containers are being replaced.

### SUMMARY

In some aspects, the techniques described herein relate to a scent dispensing apparatus, including: a scent container retaining apparatus configured to receive and securely retain a scent container, wherein the scent container includes a smart vial tag; a smart vial sensor directionally positioned to have a direct line of sight with the smart vial tag to read the smart vial tag when the scent container is securely retained in the scent retaining apparatus; and a fragrance management application that receives an identity of the smart vial tag from the smart vial sensor and generates a unique container profile for the scent container.

In some aspects, the techniques described herein relate to a scent dispensing apparatus, wherein the smart vial sensor is an optical sensor and the smart vial tag is a QR code detectable by the smart vial sensor.

In some aspects, the techniques described herein relate to a scent dispensing apparatus, wherein the smart vial sensor is a radio receiver and the smart vial tag is an RFID tag that can be read by the radio receiver.

In some aspects, the techniques described herein relate to a scent dispensing apparatus, wherein the radio receiver is a directional antenna positioned within a threshold distance of the RFID tag when the scent container retaining apparatus retains the scent container.

In some aspects, the techniques described herein relate to a scent dispensing apparatus, wherein the unique container profile includes one or more of an identity of a scent solution, settings of a scent solution, states of the scent container, a fill-level of the scent container, and a location of the scent container.

In some aspects, the techniques described herein relate to a scent dispensing apparatus, wherein the scent container includes a scent solution that can be emitted from the scent container.

In some aspects, the techniques described herein relate to a scent dispensing apparatus, wherein the smart vial tag further includes smart container data that can be read from the smart vial tag.

In some aspects, the techniques described herein relate to a scent dispensing apparatus, wherein the smart vial tag is located on a bottom surface of the scent container.

In some aspects, the techniques described herein relate to a scent dispensing apparatus, wherein the unique container profile includes historical data of the scent container.

In some aspects, the techniques described herein relate to a method of using a scent dispensing apparatus, including: detecting, using a smart container sensor of a scent dispensing apparatus, that a scent container with a smart container tag positioned on a surface of the scent container has been positioned in a slot of the scent dispensing apparatus; receiving, using the smart container sensor, smart container data from the smart container tag; determining, using a processor of the scent dispensing apparatus, an identity of the scent container using the smart container data; determining, using the processor of the scent dispensing apparatus, current usage data of the scent container; and updating, using the processor of the scent dispensing apparatus, the smart container data to include the current usage data by having the smart container sensor provide current usage data to the smart container tag of the smart container.

In some aspects, the techniques described herein relate to a method, wherein the smart container sensor is a radio sensor and the smart container tag is a RFID tag that can be read by the radio sensor when the RFID tag is within a threshold distance of the radio sensor.

In some aspects, the techniques described herein relate to a method, wherein the RFID tag is positioned on a bottom surface of the scent container and the radio sensor is positioned below where the scent container is positioned in the scent dispensing apparatus.

In some aspects, the techniques described herein relate to a method, wherein the radio sensor is a directional antenna and wherein when the scent container is positioned in the scent dispensing apparatus, the radio sensor has a direct line of sight with the RFID tag.

In some aspects, the techniques described herein relate to a method, wherein the smart container data includes a unique identifier that represents the identity of the scent container.

In some aspects, the techniques described herein relate to a method, wherein the smart container sensor is an optical sensor and the smart container tag is a QR code that is detectable by the optical sensor.

In some aspects, the techniques described herein relate to a method, wherein the smart container sensor includes electrical sensor contacts configured to engage with electrical tag contacts on the smart container tag.

In some aspects, the techniques described herein relate to a method, wherein the scent container includes a scent solution that can be emitted from the scent container.

In some aspects, the techniques described herein relate to a method, wherein the current usage data includes one or more of an identity of a scent solution, settings of a scent solution, states of the scent container, a fill-level of the scent container, and a location of the scent container.

In some aspects, the techniques described herein relate to a method, wherein updating the smart container data to include the current usage data further includes: tracking the smart container tag during a manufacturing process to include first tracking data; tracking the smart container tag during a shipping process to include second tracking data; and updating the smart container data to include the first tracking data and the second tracking data.

In some aspects, the techniques described herein relate to a smart container tag tracking method including: installing a smart container tag with a unique identifier on a scent container during manufacturing; tracking the smart container tag during a manufacturing process; tracking the smart container tag during a shipping process; automatically connecting the smart container tag to a scent dispenser using a smart container sensor; updating a scent container lifetime based on the tracking of the smart container tag; applying a timestamp at removal of the scent container; and updating the smart container tag with the timestamp.

Further innovative aspects include corresponding systems, methods, apparatus, and computer program products. These systems, methods, apparatus, computer program products, and other aspects, are particularly advantageous in a number of respects. For example, the technology enables users to remotely control scent dispensers, switch between scents remotely, and receive notifications when a scent solution is running/has run out, automatically switches the scent solution(s) to diffuse based on user location, allows users to schedule scent diffusion for different locations, etc. In addition, the technology can collect data about scent solution usage and user habits when using scent dispensers, and then analyze that data to produce analytics to inform producers about characteristics of the users, the scents being consumed, etc. Numerous additional features are also possible and contemplated, many of which are discussed herein.

However, this list of features and advantages is not all-inclusive and many additional features and advantages are within the scope of the present disclosure. Moreover, it should be noted that the language used in the present disclosure has been principally selected for readability and instructional purposes, and not to limit the scope of the subject matter disclosed herein.

### BRIEF DESCRIPTION OF THE DRAWINGS

FIG. 1A is a block diagram illustrating an example system for scent dispensation with smart container detection.

FIG. 1B is a block diagram illustrating an example scent dispenser with smart container detection.

FIGS. 2A-2B are example scent dispensers with smart container detection.

FIGS. 3A-3B are examples scent dispensers with smart container detection.

FIGS. 4A-4B are examples of a smart container detection system of an example scent dispenser.

FIG. 5 is a flowchart of an example method of smart container detection.

FIG. 6 is a block diagram of an example smart container tracking system.

### DETAILED DESCRIPTION

The technology described in this disclosure relates to detecting scent containers using a smart container detection system for dispensing scent within the premises using remotely controllable scent dispensers. As an example, the technology allows the user to install a scent container into a scent dispenser and the scent dispenser is able to automatically detect an identity of the scent container and/or various settings and/or states of the scent container, such as a fill-level of the scent container after the scent container is installed in the scent dispenser without requiring any additional data input from the user.

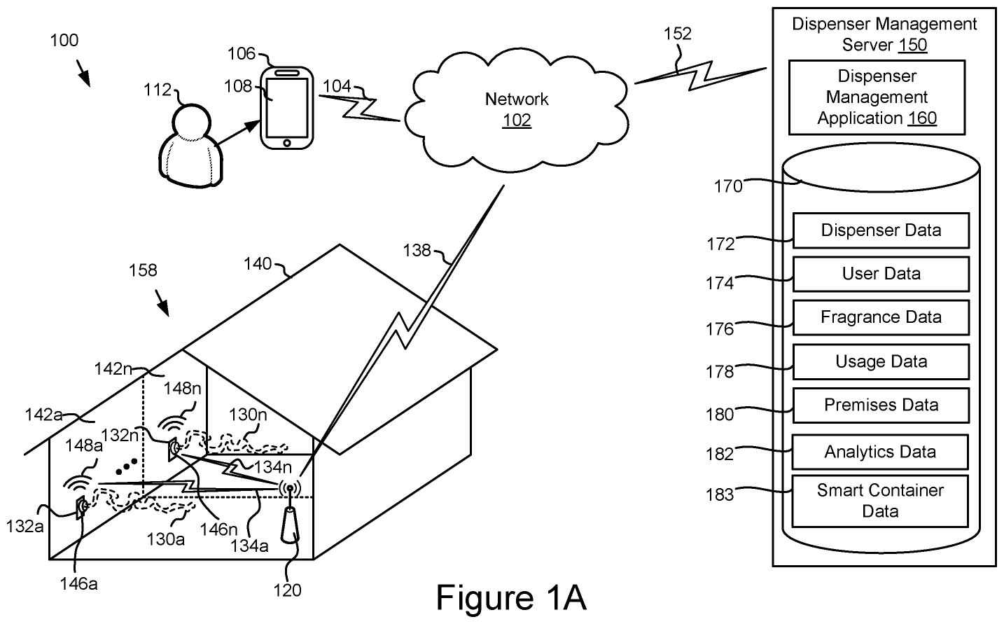

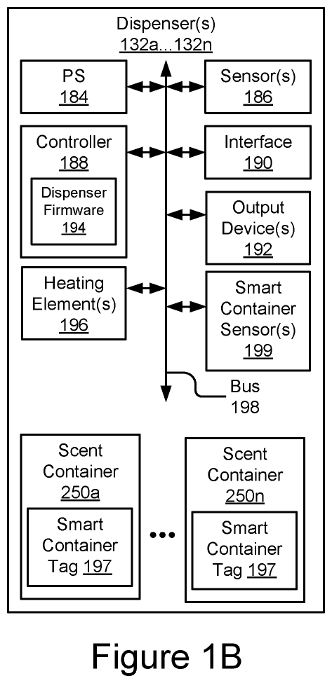

FIG. 1A is a block diagram illustrating an example system 100 for smart container detection and scent dispensation. The system 100 may include one or more scent dispenser installations 158. In a typical implementation, a multiplicity of users 112 may install scent dispenser on their respective premises 140 in which they wish to dispense various scents. A scent dispenser installation 158 may include any number of scent dispensers 132a . . . 132n. In the depicted implementation, scent dispensers 132a and 132n (individually or collectively also referred to as simply 132) are plugged into electrical sockets 146a and 146n. The scent dispensers 132 may be located in the same room or in different rooms (e.g., 142a and 142n).

As shown, the illustrated system 100 further includes client device(s) 106 and a dispenser management server 150, which are electronically communicatively coupled via a network 102 for interaction with one another, access point(s) 120, and the scent dispenser(s) 132, etc., using standard networking protocols, as reflected by signal lines 104, 138, and 152. In a typical installation, the scent dispenser(s) 132 are coupled for electronic communication with the access point(s) 120 (e.g., a modem, router, etc., coupled to the network 102) as reflected by the signal line 138. In further embodiments, the scent dispenser(s) 132 may be coupled directly to the network 102, such as via a wireless wide area network (e.g., WWAN) or other suitable network. For clarity, reference to the term network 102 includes the local networks of the installations (e.g., provided by access point(s) 120, etc.), unless otherwise specified.

The scent dispenser 132 includes a communication-enabled diffusion device that diffuses scent solution(s) from scent container(s) installed in the scent dispenser 132. The scent dispenser 132 may include wireless transceivers configured to communicate with other devices within range, such as other scent dispenser(s) 132 and/or access point(s) 120. In some embodiments, the diffusion device nebulizes the scent solution into the air of the surrounding environment to diffuse the scent. In some implementations, the diffusion device may nebulize the scent solution into the air using diffusion elements such as heating elements, fan elements, or other elements that can cause a scent solution or scent gel capsule to be emitted as a scent as shown by the emitted scent lines 130a-130n. The structure, acts, and/or functionality of the scent dispenser 132 is discussed further below. In some implementations, the scent dispenser 132 may include a smart container detection system for detection of scent container(s) being installed in the scent dispenser 132.

In some implementations, the access point 120 connects to the set of scent dispensers 132 installed on the premises 140, as shown by the signal lines 134a . . . 134n, and exchanges data sent between the scent dispensers 132 and the mobile device(s) 106 and/or the dispenser management server 150 (e.g., via the signal line 138). In further implementations, the scent dispensers 132 may be connected directly to the network 102 without needing the access point 120.

The dispenser management application 160 operable by the dispenser management server 150 can receive operational data from the scent dispensers 132 in association with the premises 140, room 142, user(s) 112, and/or scent containers with which they are associated. The dispenser management application 160 can receive management requests and geolocation data from mobile devices of the user(s) 112 in association with the scent dispenser(s) 132 that are registered to the user(s) in the system 100. The management requests may include registration data requesting to register a scent dispenser 132 with a user's 112 account, a particular premises 140, and/or a room 142 within the premises 140; scheduling data requesting to schedule dispensation of scent(s), from scent dispenser 132 or a set of two or more scent dispenser(s) 132, for particular timeframe(s) within a particular room 142 of a premises 140; operational data instructing to activate or deactivate a scent dispenser 132 and/or specifying which scent solution to activate and/or deactivate; geolocation data indicating a current location of a user 112, etc. The scheduling data may include a reoccurring schedule that repeats on certain days and/or times of day, and may specify which scent solution(s) should be dispensed at which times.

In some embodiments, the dispenser management application 160 may maintain the device states, scent allocations, users, and schedules of the scent dispensers 132. The dispenser management application 160 may make device function calls to command/control the scent dispensers 132 (e.g., by sending state settings), and may receive and log published events (heartbeat, alerts, data, etc.) received from the scent dispensers 132 in the data store 170.

The dispenser management server 150 includes a data store 170 storing various types of data used by the dispenser management application 160. Example data types include dispenser data 172, user data 174, fragrance data 176, usage data 178, premises data 180, analytics data 182, and/or smart container data 183. The dispenser data 172 may include entries for the scent dispensers 132 in the system 100. A given entry may include a unique identifier of the corresponding scent dispenser, a firmware version, an operational status indicator (e.g., on, off, etc.), a schedule including which day(s) of the week and/or times of day the scent dispenser should be operating, which scent solution(s) should be dispensed during the scheduled timeframe(s), whether/which color light should be illuminated, the user identifier(s) with which the scent dispenser is registered, etc.

The user data 174 may include entries for the users 112 of the system 100. A given entry may include a unique identifier for the user, contact information for the user (e.g., address, phone number, electronic address (e.g., email)), payment information, scent subscription information specifying which reoccurring scent containers should be shipped to the user, historical scent container purchase information, etc.

The fragrance data 176 may include entries for the different scent solutions that are supported by the system 100. An example entry may include a unique identifier for a given scent, a scent name, a scent description, a list of ingredients that comprise the scent's composition, an indication of the strength of a scent, room types for which to suggest the scent, etc.

The usage data 178 may include logged usage statistics received from the scent dispensers 132 deployed in the system 100. Example usage statistics may include hours of operation, which scent solutions were dispensed during those hours of operation, thus reflecting the time of day scent solutions were utilized, the amount of scent solutions that were dispensed as measured by container sensors included in the scent dispensers 132, the rate at which the scent solutions were dispensed, etc. The usage statistics for a given scent dispenser 132 may be specifically associated with the user 112 associated with the usage and/or a room 142 of a premises 140 in which the scent dispenser 132 is installed.

Further, the fragrance data 176 may include container data including unique identifiers for each scent container produced and registered for use in the system 100. The container data may reflect the installation status of a container, the unique identifier of the scent solution contained by the container, and the fill level of the container, an eligibility status, etc. For instance, the container data for a particular container may indicate whether the container is new and has not yet been installed, that the container is currently installed but has not yet been depleted of its solution, that the container was depleted of its solution and is no longer eligible for use, and/or that the container has been refilled and is available for future use or to be shipped back out to customers.

The premises data 180 may include entries for different premises 140 in which scents dispensers 132 are installed. The premises data 180 may include different rooms 142 for the premises 140, and may indicate in which specific rooms 142 the scent dispensers 132 are installed. Example premises data may include unique identifiers for each of the premises, room names for each of the rooms of a given premises (e.g., which may be customized by the user), the dimensions of the rooms of the premises (which may provided by users 112 or automatically determined based on estimated room dimensions, blueprints, etc.), an indication of which scent dispensers 132 are installed in which rooms of a given premises, for example, using the unique identifiers of the scent dispensers 132, etc., an indication of which unique user identifiers are associated with which unique premises identifiers, etc.

The analytics data 182 may include data produced by the dispenser management application 160 the data stored in the data store 170. For instance, data from the scent dispensers belonging to users 112 may be aggregated by the server in the data store 170 and then analyzed to generate analytics. These analytics can be used by the manufacturer to improve the product, the user to evaluate his/her usage, and the scent solution producers to learn more about their users, the users' habits and preferences, their products, etc.

Example analytics data may reflect scent preferences, such as which scents consumers use most often (e.g., based on the usage data 178 determined by a container sensor), scent preferences by room 142 type of the premises 140 (e.g. floral for bathrooms, edible scents for kitchens, etc.) (e.g., based on the correlation between the fragrance data 176 and the premises data 180), which scents consumers are repurchasing the most (based on user data 174), whether or not consumers are mixing scents in specific rooms (e.g., based on the correlation between the fragrance data 176, the usage data 178, and the premises data 180), scent preferences by season (based on time/data coded usage data 178), preferences for strength of a scent (light vs. strong fragrance) and does that differ by room (e.g., based on the correlation between the fragrance data 176, the usage data 178, and the premises data 180), whether consumer scent preferences evolve over time (e.g., based on the usage data 178), preference for classic vs. novelty scents (e.g., based on the usage data 178 and the fragrance data 176 that tags each scent as classic or novelty), etc.

Example analytics data may reflect scent dispensation behaviors, such as how often users use their scent dispensers 132 (e.g., based on the usage data 178), is the use continuous or as needed (e.g., based on the usage data 178), how long is the continuous use (e.g., several hours, e.g., several days, etc., based on the usage data 178), how long does it take for consumers to change out a scent solution/cartridge/refill (e.g., usage data reflects an empty container stayed in the scent diffuser 132 for a certain timeframe, which can be averaged over many, or all users (e.g., generally, for that fragrance type, etc.)), how often are consumers purchasing refills (e.g., based on the user data 174 reflecting purchase history), preferred rooms 142 for using scent dispenser 132 (e.g., based on the correlation between the usage data 178 and the premises data 180), use of scent dispensers 132 when users 112 are in vs. out of the premises 140 (e.g., based on the correlation between the users' 112 user data 174 and the usage data 178), etc.

Example demographics data may reflect behaviors and preferences by different demographic groups. User data may reflect a user's gender, age, income, race/ethnicity, number of children in the home, number of pets in the home, region, settlement-type (urban, rural, suburban, etc.), premises type (single family home, apartment, condo, townhouse, small business, large business, hotel, bed and breakfast, warehouse, restaurant, etc.), personal or business, etc. Using the user data, any of the other data or combinations thereof may be segmented to determine which groups may be more or less predisposed to scent dispensation, and how different factors affect the level of scent dispensation by a particular group. In some implementations, the demographic data may be used to provide recommendations to users 112 based on various data. In further implementations, the recommendations may be determined using artificial intelligence and/or machine learning to analyze the demographic data and identify various trends for recommendations.

The smart container data 183 may include entries for different scent containers installed in scents dispensers 132 that are identified by the smart container detection system. The smart container data 183 may include one or more of identities of each of the smart containers, fill levels of the smart containers, persistent scent settings for each scent container, refill status of each scent container, historical data of each scent container, ages of each scent container, etc. In some implementations, the smart container tag 197 may include a unique identifier that is associated with smart container data 183 stored on the dispenser management server 150 and the smart container data 183 may be updated based on changes in the settings or details associated with the smart container and based on the unique identifier of the smart container tag 197. In some implementations, the smart container data 183 may be further stored on the smart container tag 197 (see FIG. 1B) and may travel with the scent container 250 rather than being stored on the dispenser management server 150.

The data stored by the data store 170 may be correlated using various common keys, such as unique identifiers (e.g., user identifiers, room identifiers, fragrance identifiers, scent dispenser identifiers, container identifiers, room identifiers, premises identifiers, smart container tag 197 identifiers, etc.), which allow the dispenser management application 160 to generate and provide rich analytics.

The container sensors of the scent dispensers 132 can detect the changes in the fluid levels of the containers as the solutions from the containers are diffused into the air of the respective premises 140. The controllers of the scent dispensers 132 receive the data reflecting the changing fluid levels and transmit that data to the dispenser management server 150, which in turn stores the data as usage data 178 in the data store 170 in association with the scent/fragrance ID to which the data corresponds. As a result, the usage data 178 reflects the amount of each scent solution that is consumed over time for each scent dispenser 132 relative to the user ID, the premises ID, the room ID, scent dispenser 132 ID, the container ID associated with that particular scent solution (e.g. which is identifiable as discussed elsewhere herein).

FIG. 1B is a block diagram illustrating an example scent dispenser, which is depicted as including a power supply (PS) 184, a set of sensors 186, a controller 188, an interface 190, output device(s) 192, dispenser firmware 194, heating element(s) 196, smart container sensor(s) 199, and any number of containers 250a . . . 250n (individually or collectively also referred to as simply 205) that may each include a smart container tag 197. The components 184, 186, 188, 190, 192, 196, and 199 are communicatively coupled via a communications bus 198. The controller 188 may include a non-transitory memory device, or may be coupled to a non-transitory memory device also coupled for communication via the bus 198. The non-transitory memory device may store software that specially configures the controller, such as the dispenser firmware 194. The PS 184 may be any AC and/or DC power supply for powering the scent dispenser 132. The controller 188 may be a microchip that controls the constituent electronics (e.g., sensor(s) 186, output device(s) 192, interface 190, heating element(s) 196, etc.) of the scent dispenser 132.

The set of sensor(s) 186 may include temperature sensors (e.g., thermocouples, etc.) for sensing the temperature of the heating element(s) 196, ambient light sensor to detect a light level in a surrounding environment (e.g., room), and/or a motion sensor to detect motion in the surrounding environment, etc.

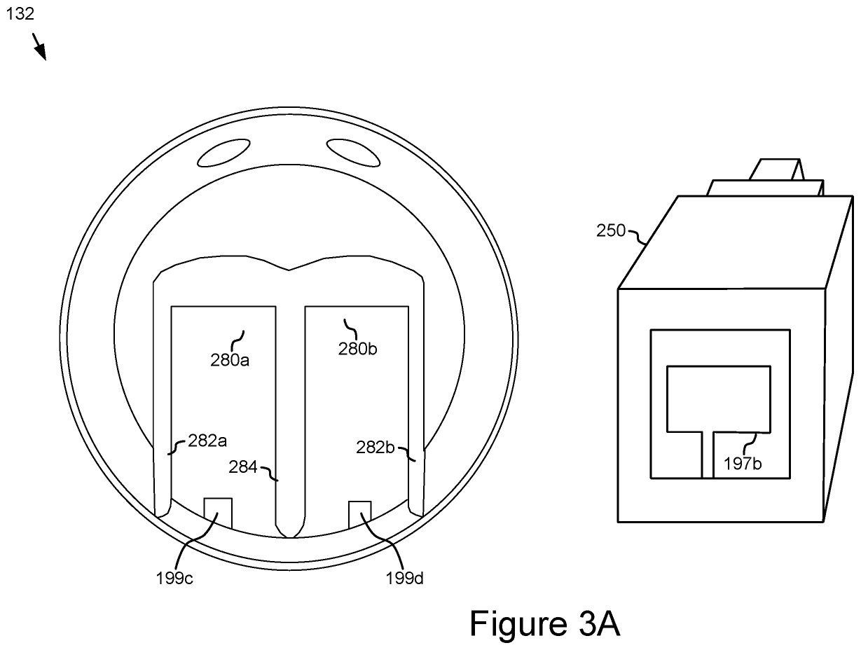

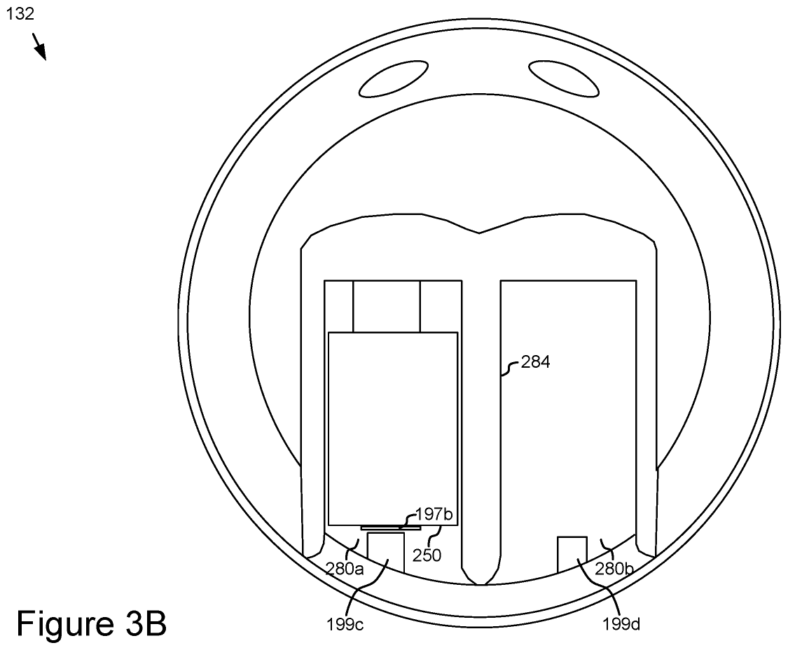

In some embodiments, the sensors 186 may include a temperature bay sensor for each heating element 196 (e.g., that may measure temperatures ranging from (−50 to 200 degrees Celsius), the container sensor 300 (e.g., see FIG. 3A), an ambient light sensor, etc.). The heating element 196 may be configured to heat to any suitable temperature sufficient to diffuse a scent solution contained in a corresponding container 250, a non-limiting example of which may be 65 degrees Celsius, and the temperature bay sensor may be mounted on or adjacent to, or embedded in, the heating element 196 to measure the temperature of the heating element 196 and provide feedback to the controller 188, which may cyclically heat the heating element 196 based on the feedback to maintain a constant or substantially constant temperature during diffusion (as set in the state settings).

The sensors 186 include a transceiver having a wireless interface configured to communicate with the devices coupled to the network 102, such as the access point 120, the dispenser management server 150, and/or other components of the network 102 using standard communication protocols, such as Internet protocols. Further, the transceiver may be configured to wirelessly transmit data via a meshwork network made up of a plurality of scent dispensers 132 and/or other devices, such as the access point 120 or a mobile device 106. By way of further example, the transceiver may transmit data to the access point 120 to which it is linked using a protocol compliant with IEEE 802.15, such as Zigbee®, Z-Wave®, Bluetooth®, or another suitable standard. Additionally or alternatively, one or more of the scent dispenser(s) 132 and/or the access point 120 of an installation 158 may be wired for direct communication and the wired components may exchange data using wired data communication protocols. Further embodiments are also possible and contemplated. In some embodiments, the transceiver may be embedded in the controller 188 or may be a component distinct from the controller and coupled to the controller 188 via the bus 198.

The smart container sensor(s) 199 may include one or more of an optical sensor for detecting a specific smart container tag 197 of a scent container 250 that has an unique image or pattern (such as a QR code) detectable by the optical sensor, a container sensor for sensing the fill level of the container(s) 250 installed in the scent dispenser 132, a radio antenna that can detect a smart container tag 197, such as an RFID tag, or any other sensor that can detect a smart container tag 197 in proximity to the smart container sensor(s) 199. In some implementations, the smart container sensor(s) 199 may be referred to as a reader and may be configured to read wireless electrical signals, such as from an RFID or other similar transmitting device.

The output device(s) 192 may include light sources and/or audio reproduction devices, although further suitable output devices are also contemplated and applicable. The light sources and/or audio reproduction devices may be controlled to produce output consistent with a scent being emitted by the scent dispenser (e.g., a low, soothing light and music may be output in conjunction with a relaxing scent being emitted).

Returning to FIG. 1A, the client device(s) 106 (also referred to individually and collectively as 106) are computing devices having data processing and communication capabilities. In some embodiments, a client device 106 may include a processor (e.g., virtual, physical, etc.), a memory, a power source, a network interface, and/or other software and/or hardware components, such as a display, graphics processor, wireless transceivers, keyboard, camera, sensors, firmware, operating systems, drivers, various physical connection interfaces (e.g., USB, HDMI, etc.).

The client devices 106 may couple to and communicate with one another and the other entities of the system 100 via the network 102 using a wireless and/or wired connection. Examples of client devices 106 may include, but are not limited to, mobile phones (e.g., feature phones, smart phones, etc.), tablets, smartwatches or other smart wearables, laptops, desktops, netbooks, server appliances, servers, virtual machines, TVs, set-top boxes, media streaming devices, portable media players, navigation devices, personal digital assistants, etc. In addition, while a single client device 106 is depicted in FIG. 1A, it should be understood that any number of client devices 106 may be included.

As shown, the client device 106 may include a scent application 108, which allows the user to set scent dispenser 132 settings, turn scent dispensers 132 on and off, purchase containers for the scent dispenser 132, set up a scent dispenser 132, register an account, set up a premises and the rooms of the premises, associate a scent dispenser 132 with a particular room of the premises, view analytics reflecting the user's historical use of his/her scent dispenser(s) 132, enable user profiles to use and setup scent profiles for the scent dispenser(s) installed in the premises, set a profile hierarchy (e.g., set which user profile(s) is/are the dominant user profile), etc.

The client device 106 may store the scent application 108 in non-transitory memory, retrieve the scent application 108 from memory, and execute instructions comprising the scent application 108. The scent application 108, when executed by a processor of the client device 106, configures the processor of the client device 106 to carry out the acts and functionality described herein. In some embodiments, the scent application 108 may render and display various interfaces for carrying out the functionality described herein.

Returning to FIG. 1A, the dispenser management server 150 may include one or more computing devices having data processing, storing, and communication capabilities. For example, the server 150 may include one or more hardware servers, virtual servers, server arrays, storage devices and/or systems, etc., and/or may be centralized or distributed/cloud-based. In some embodiments, the server 150 may include one or more virtual servers, which operate in a host server environment and access the physical hardware of the host server including, for example, a processor, memory, storage, network interfaces, etc., via an abstraction layer (e.g., a virtual machine manager).

While not depicted, the server 150 may include a (physical, virtual, etc.) processor, a non-transitory memory, a network interface, and a data store 170, which may be communicatively coupled by a communications bus. Similarly, the client device 106 may include a physical processor, a non-transitory memory, a network interface, a display, an input device, a sensor, and a capture device. It should be understood that the server and the client device may take other forms and include additional or fewer components without departing from the scope of the present disclosure.

Software operating on the server 150 (e.g., the dispenser management application 160, an operating system, device drivers, etc.) may cooperate and communicate via a software communication mechanism implemented in association with a server bus. The software communication mechanism can include and/or facilitate, for example, inter-process communication, local function or procedure calls, remote procedure calls, an object broker (e.g., CORBA), direct socket communication (e.g., TCP/IP sockets) among software modules, UDP broadcasts and receipts, HTTP connections, etc. Further, any or all of the communication could be secure (e.g., SSH, HTTPS, etc.).

As shown, the server 150 may include a dispenser management application 160 embodying a remotely accessible scent service. The dispenser management application 160 may send data to and receive data from the other entities of the system including the controllers 188 and/or 328, the mobile device(s) 106, etc. The dispenser management application 160 may be configured to store and retrieve data from one or more information sources, such as the data store 170. In addition, while a single server 150 is depicted in FIG. 1A, it should be understood that one or more servers 150 may be included.

In some embodiments, the dispenser firmware 194, the scent application 108, the dispenser management application 160, etc., may require users to be registered to access the acts and/or functionality provided by them. For example, to access various acts and/or functionality provided by the scent application 108, dispenser management application 160, and/or scent dispensers 132, these components may require a user to authenticate his/her identity (e.g., by confirming a valid electronic address). In some instances, these entities 108, 132, 150, etc., may interact with a federated identity server (not shown) to register/authenticate users. Once registered, these entities 108, 132, 150, etc., may require a user seeking access to authenticate by inputting credentials in an associated user interface.

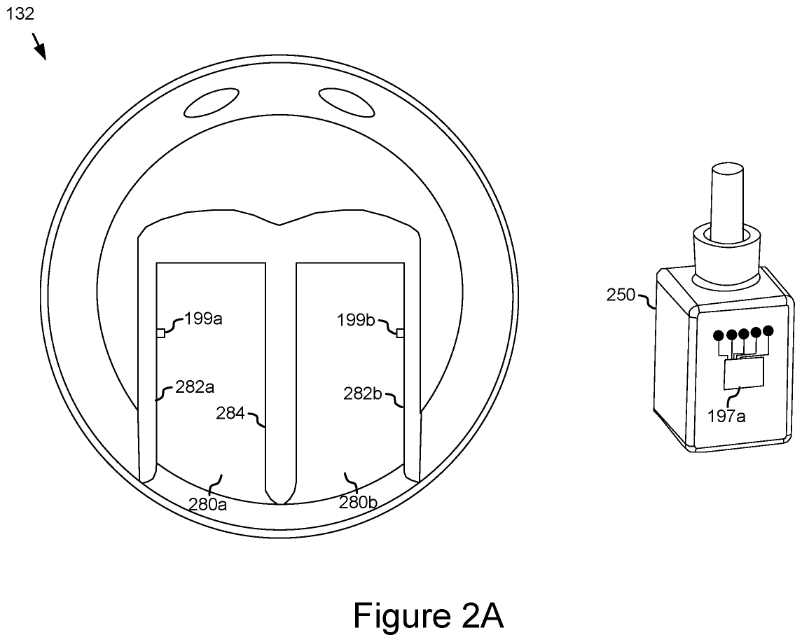

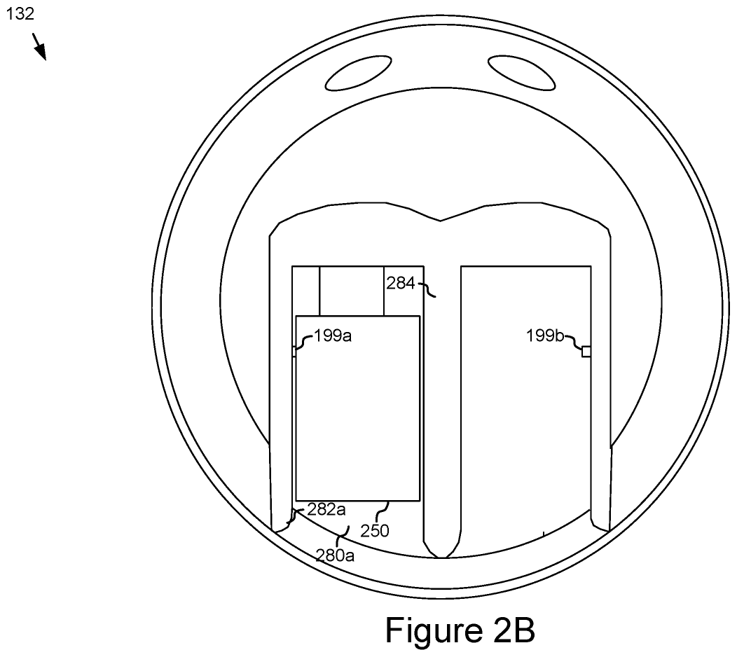

FIG. 1B includes one or more scent containers 250a-250n. although it should be understood that the container 250 may take a variety of different forms beyond what is described with respect to FIGS. 2-4 and have different dimensions. The scent containers 250 may include a scent solution that may include chemical(s) that are processed by the scent diffuser 132 to diffuse or nebulize it within the room in which the scent dispenser 132 is placed. The solution in some embodiments may be a liquid perfume, an essential oil, a scented gel, or other suitable solutions. In some alternative, the perfume or essential oil may be caked and the caked substrate may be heated to diffuse the scent. The scent container 250 may be made of any suitable material, such as plastic, metal, ceramic material, glass, etc.

In some embodiments as described with respect to FIGS. 2-4, a container 250 (e.g., a first container, second container, etc.) may include a first side, one or more sidewalls, and a second side, such as to form a rectangular shape with a cavity in the center in which a scent solution may be held. In other embodiments, the container 250 may be cylindrical in shape with a hollow center that may hold the scent solution. In an example, a container 250 having a tubular sidewall may be a continuous around a circumference. In another example, a container 250 having a square, triangular, rectangular, etc., cross-section (e.g., halfway between the second side and the first side) at a middle of the sidewall, may have a plurality of sidewalls that are joined along at the corners of the cross-section. It should be understood that the container 250 may have any shape and is not limited to the shapes and configurations described herein. The one or more sidewalls connecting the first side to the second side. The first side, the one or more sidewalls, and the second side collectively form an inner cavity in which a scent solution is stored. The first side may include an opening through which a solution containable in the inner cavity can be dispensed from the container 250. In some embodiments, the first side may include a neck forming the opening, and the sidewall(s) and second side form a hollow body. The hollow body may form a cavity containing the scented solution. In some implementations, a wick or other dispersal element may extend from the cavity through the opening of the neck.

In some embodiments, the container 250 may include a stopper and a wick. The stopper may insert into an opening of the container 250 and forms a seal against an inner surface of the neck or other surface of the body of the container 250 in order to keep scent solution from leaking other than through the dispersal element. The stopper includes a through-hole extending through a center portion of the stopper from an outer surface of the stopper to an inner surface of the stopper. The through-hole/opening is formed in the stopper to allow a wick inserted into the cavity of the container to extend outwardly from the through-hole, and thus the opening of the first side.

In some implementations, as shown with respect to FIGS. 2-4, the container 250 may include a retaining mechanism and the retaining mechanism of the container 250 couples with a corresponding retaining mechanism included in a slot of the scent dispenser 132. In some implementations, the retaining mechanism may be a protrusion extending outwardly from a surface of the container, such as the neck or the body of the container. In the specific example depicted, the protrusion extends around the circumference of the neck of the container 250, and the retaining mechanism includes one or more coupling devices that engage with the retaining mechanism. In some embodiments, the coupling devices may comprise one or more fasteners formed to detachably engage with the neck of the container, while in other embodiments, the container 250 may sit within the container 250 without being held by a retaining mechanism.

It should be understood that other variations for the retaining mechanism are also possible and contemplated. For instance, the protrusion may engage with corresponding channels formed in a surface of the retaining mechanism into which protrusion slides into an opposing side(s). In another example, the neck includes a grove and the retaining mechanism includes corresponding protrusions or rails that mate with the grove. Numerous other variations are also possible and contemplated.

As illustrated in FIG. 1B the scent dispenser 132 may include a heating element(s) 196 to heat the solution from the container(s) 250 installed in the scent dispenser 132. In some embodiments, the wick passes a scented solution to the heating element 196. The heating element 196 diffuses the scented solution by heating the scented solution to the temperature level regulated by the controller 188.

An example heating element 196 may include a ceramic material (e.g., ceramic resistor, such as a wire wound ceramic heating element). Heating the solution received from the container 250 accelerates diffusion of the solution into the surrounding air. The housing (not shown) may include one or more ports in the lid to allow the diffused air to pass into the surrounding environment. The more heat applied by the diffusion device, the stronger the evaporative rate and thus the stronger the smell that may be produced.

In some embodiments, a scent solution of a container 250 may require a certain heating range to be diffused effectively, and the controller 188 may receive specific state settings for that scent solution from the dispenser management application 160, which in some implementations may be provided by the identity of the scent container 250 found on the smart container tag 197 and use those settings to control the temperature of the corresponding heating element 196.

FIG. 2A is an example scent dispenser 132. As shown in FIG. 2A, the scent dispenser 132 may have a removable outer housing (not shown) and the internal components may be configured for a scent container 250 to be installed in the scent dispenser 132. As shown, in some implementations, a two scent container configured scent dispenser 132 may include a first slot 280a and a second slot 280b to allow for multiple different scent containers 250a-250n to be installed. It should be understood that while two slots 280 are shown in FIG. 2A, any number of slots 280 to allow for any number of scent containers 250 are contemplated, such as a single scent container slot or a multi-scent container scent dispenser 132, with two scent container positions, three scent container positions, four scent container positions, etc. to allow multiple scent containers 250 to be positioned within the multi-container scent dispenser 132. In some implementations, the slots 280 are formed out of a sidewall 282 on one side and a partition member 284 on the other side. In some instances, the partition member 284 may separate or divide the different slots 280, while in other implementations, a partition member 284 may not be present and each slot 280 is an area in which a scent container 250 can be positioned without the partition member 284. In some implementations, when a scent container 250 is positioned within the slot 280, the partition member 284 may apply pressure against the side of the scent container 250 in order to cause the scent container 250 to be retained within the slot 280. It should also be understood that in a single scent container 250 implementation, the partition member 284 may be absent and both sidewalls 282 may form the slot 280 in which the scent container 250 may be positioned.

As shown in FIG. 2A, the scent dispenser 132 may include one or more smart container sensors 199a and 199b. The position of these smart container sensor(s) 199a-199b allows the smart container sensor(s) 199a-199b to be to be positioned proximate to scent container(s) 250 such that the smart container tag(s) 197 of each of the scent container(s) 250 may be readable by the smart container sensor(s) 199a-199b, such as by being electrically connected to the smart container sensor(s) 199a-199b. In this example, the smart container sensor(s) 199a-199b may be electrical contacts configured to interact with corresponding electrical contacts on the smart container tag 197a as shown. The corresponding electrical contacts of the smart container tag 197 may be part of a circuit forming the smart container tag 197. The circuit of the smart container tag 197 may store various smart container data 183 directly on the circuit, such as an identity of the scent solution in the scent container 250, a fill level of the scent container 250, usage data of the scent container 250, previous locations of the scent container 250, refill data of the scent container 250, order/purchase data of the scent container 250, etc.

As shown in FIG. 2B, as the scent container 250 is inserted into the slot 280a and contact is made between the respective contacts of the smart container sensors 199a and the smart container tag 197 (not shown as the left side of the scent container is not visible in the front vial as shown), the controller of the apparatus can detect the smart container data 183, such as an identity of the smart container 250, an identity/type of the sent solution, ingredients of the scent solution, etc. based on signals from the vial circuit and the smart container tag 197. The controller 188 of the scent device 132 can read information from the smart container tag 197, such as an identity of the scent container 250, the amount of scent that the scent container contains, etc. based on signals from the smart container tag 197. For example, the scent container tag 197 may be configured to store smart container data 183 identifying the scent solution (e.g., a unique identifier associated with the scent solution), ingredients of the scent solution, an identifier of the scent container 250, etc. and the controller 188 may access a lookup table or other similar functionality to determine which scent it is. The circuit of the smart container tag 197 may also store information about when the scent solution/container was manufactured, and how long the container has been used (such as how long a scent has been dispensed from the scent container 250). For example, the controller 188 can write to the circuit (a memory thereof) of the smart container tag 197 usage data reflecting how long the scent container 250 has been used. If a user 112 removes a scent container 250 and then replaces it later, the controller 188 can detect how much solution is left/available for dispensation (2 hours have been used, 30 are left, and so forth) based on information provided by the scent container tag 197. In further implementations, rather than storing the smart container data 183 on the circuit, the smart container data 183 can be stored on the server 150 and the smart container tag 197 includes the unique identifier that the smart container data 183 associated with that smart container tag 197 unique identifier is associated with that smart container data 183 stored and read on the server 150.

FIG. 3A is another example scent dispenser 132. As shown in FIG. 3A, the scent dispenser 132 may have a removable outer housing (not shown) and the internal components may be configured for a scent container 250 to be installed. As shown, the scent dispenser 132 may include a first slot 280a and a second slot 280b to allow for multiple different scent containers 250a-250n to be installed. It should be understood that while two slots 280 are shown in FIG. 3A, any number of slots 280 may be included to allow for any number of scent containers 250 that may be contemplated as described elsewhere herein. Such as a single slot 280 for a single scent container 250 or multi-container scent dispensing devices 132, etc. In some implementations, the slots 280 are formed out of a sidewall 282 on one side and a partition member 284 on the other side. In some instances, the partition member 284 may separate the different slots 280. It should also be understood that in a single scent container 250 implementation or in other implementations where a partition member 284 is not present, the partition member 284 may be absent and both sidewalls 282 may form the slots 280 in which the scent containers 250 may be positioned.

As shown in FIG. 3A, the scent dispenser 132 may include one or more smart container sensors 199c and 199d. In this example, the smart container sensor(s) 199c-199d may be radio transmitters/receivers that can be configured to read a smart container tag 197b in the form of a radio transponder that can broadcast a unique scent container identifier. In some implementations, the position of these smart container sensor(s) 199c-199d allows the smart container sensor(s) 199c-199d to be to be positioned directionally proximate to scent container(s) 250 such that the smart container tag 197b of the scent container(s) 250 may be proximate to the smart container sensor(s) 199c-199d without touching the smart container sensor(s) 199c-199d in order to allow wireless signals to pass from the smart container tag 197b to the smart container sensor 199c and provide smart container data 183 to the smart container sensor(s) 199c-199d. As shown in FIG. 3A, the smart container tag 197b may be a form of antenna connected to a circuit that can read/write smart container data 183 wirelessly to and from the smart container sensor 199c. In other implementations, the smart container tag 197 may read the smart container data 183, such as a unique identifier of the scent container 250, wirelessly and then may store additional smart container data 183 on the server 150 and the smart container data 183 may be stored as being associated with the unique identifier of the smart container tag 197.

The smart container tag 197b in some implementations may be positioned on a bottom surface of a scent container 250. When positioned on a bottom surface of the scent container 250, a user 112 may not have to position the scent container 250 in a specific orientation in the slot 280, but instead may merely install the scent container 250 into the slot 280 independent of the direction the scent container 250 is rotated, the smart container tag 197b can be read by the smart container sensor 199c. This allows for a user to install the scent container 250 and the smart container tag 197c can be read independent of how the user rotates the scent container 250 when positioned in the slot 280. In further implementations, the smart container tag 197 can be positioned at other points along the scent container 250 and the smart container sensor can be correspondingly positioned in the scent dispenser 132 to read/write the smart sensor tag 197.

In some implementations, when multiple scent containers 250 are positioned, the smart container sensors 199c-199d may each directional point towards their respective smart container tags 197 on their associated smart containers 250 without picking up interference from the other smart container tags 197 positioned within the scent dispenser 132. This directionality of the smart container sensors 199 allows for interference to be reduced between the smart container sensors 199 and reduces the chances of interference interrupting the process of reading the smart container tag 197 by the smart container sensor 199. In further implementations, interference shielding, may be positioned between the different smart container sensors 199 to further reduce the chances of interference between the smart container sensors 199.

As shown in FIG. 3B, as the scent container 250 is positioned into the slot 280a and the scent container tag 197b comes within a threshold distance for reading/writing the smart container tag 197b by the smart container sensor 199c, the smart container tag 197b can begin sharing the smart container data 183 with the controller 188. For example, the scent container tag 197b may be configured to store smart container data 183 identifying the scent solution (e.g., a unique identifier associated with the scent solution), and the controller 188 may access a lookup table or other similar functionality to determine which scent it is. In another example, the controller 188 can write to the circuit (a memory thereof) of the smart container tag 197b usage data reflecting how long the scent container 250 has been used. If a user 112 removes a scent container 250 and then replaces it later, the controller 188 can detect how much solution is left/available for dispensation based on information provided by the scent container tag 197.

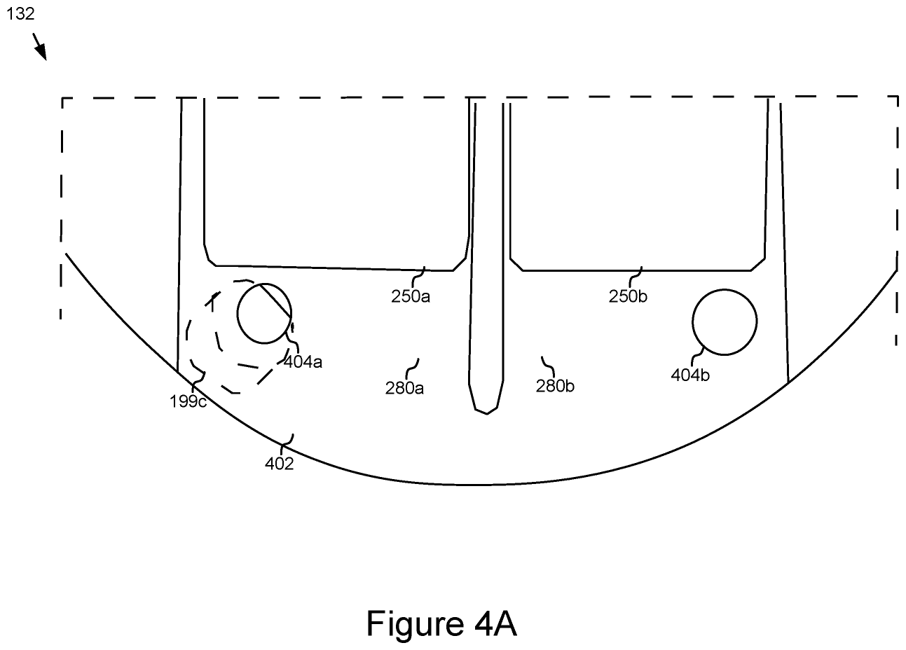

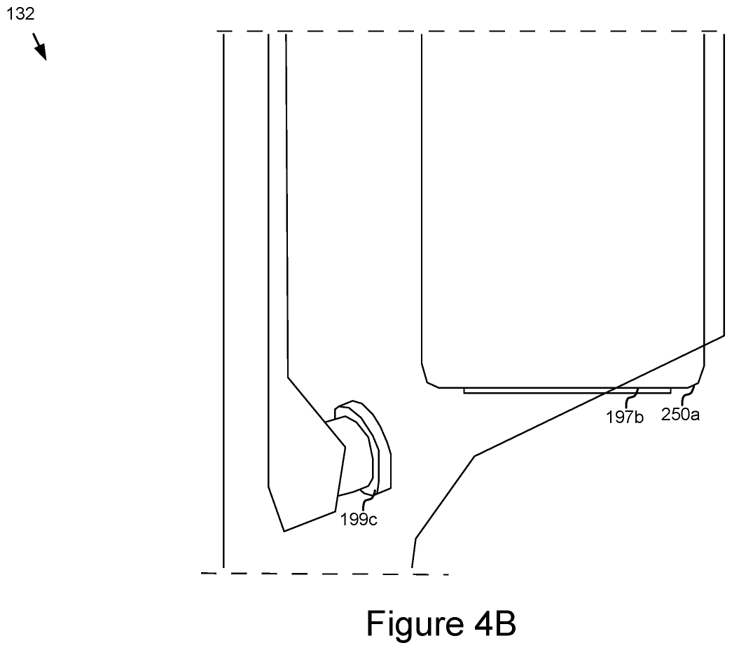

FIG. 4A depicts another example view of a portion of a scent dispenser 132. As shown, the scent dispenser 132 may include one or more scent containers 250a-250b positioned within respective slots 280a-280b. As shown, a slot housing 402 may separate the space forming the slots 280 from other components of the scent dispenser 132 (not shown , as they are hidden by the slot housing 402). In some implementations, the slot housing 402 may include one or more holes 404 or access point through which the smart container sensor(s) 199 may pass wireless signals (such as radio frequency signals) to and from the smart container tag 197 (not shown, but located on a bottom portion of the scent container 150 as shown in FIG. 4A). As shown in this example, the smart container sensor 199c (depicted in broken lines as it is hidden behind the slot housing 402) may be positioned directionally, such as at an angle below the hole 404a such that it can read the smart container tag 197 (not shown) positioned on a surface, such as a bottom surface, of the scent container 250a when the scent container 250a is positioned within the slot 280a. The hole 404a or access point allows for wireless signals to pass without interference and in some implementations, where the distance between the smart container tag 197 and the smart container sensor 199c is small enough that the smart container tag 197 and the smart container sensor 199c are almost adjacent, the slot housing 404 can act as a shield to direct the signals towards the smart container tag 197 and prohibit and unwanted signals or interference from another source from affecting what is being provided to the smart container sensor 199c. This allows the slot housing 404 to protect the smart container sensor 199 from interference.

FIG. 4B depicts another example of the smart container sensor 199c reading/writing smart container data 183 to and from the smart container tag 197b. As shown in this example, the smart container tag 197b and the smart container sensor 199c are not touching, but are proximate to each other. In some implementations, the smart container sensor 199c may include a directional antenna that allows signals to be passed directionally towards the smart container tag 197b and not pass signals in other direction to avoid interference or other unwanted data. This directionality of the antenna allows for multiple scent containers 250 with smart container tags 197 to be positioned adjacent to each other without the smart container sensor(s) 199 providing interference with each other. In some implementations, the smart container sensor 199 may be directionally positioned to have a direct line of sight with the smart container tag 197b to read the smart container tag 197b when the scent container 250 is securely retained in the scent retaining apparatus of the scent dispenser 132.

In some implementations, the scent dispenser 132 may include output devices 192 in the form of a ring of LEDs (e.g., light pipe) that are programmable to change based on a scent dispensation program. Changes may include different intensities, colors, patterns, and so forth. In some embodiments, the light patterns may reflect the state of the scent dispenser 132 (error state, instructions, low scent, change scent container, etc.). For example, if the scent container 250 on the left side is low or out, the lights on the left side may light up in red to reflect such (or if both are out both sides would illuminate in red, and so forth).

The ring of lights may be distributed equally around the front of the apparatus or in other suitable ways. On a backside of the front cover, the cover may include contacts that connect to contacts on the front of the front of the inside body of the apparatus when the cover is attached (e.g., magnetically or via other fasteners) to the inside body. As such, the contacts between the cover and the inside body decouple when the cover is removed, automatically turning the LEDs off (if on), or back on when the cover is replaced.

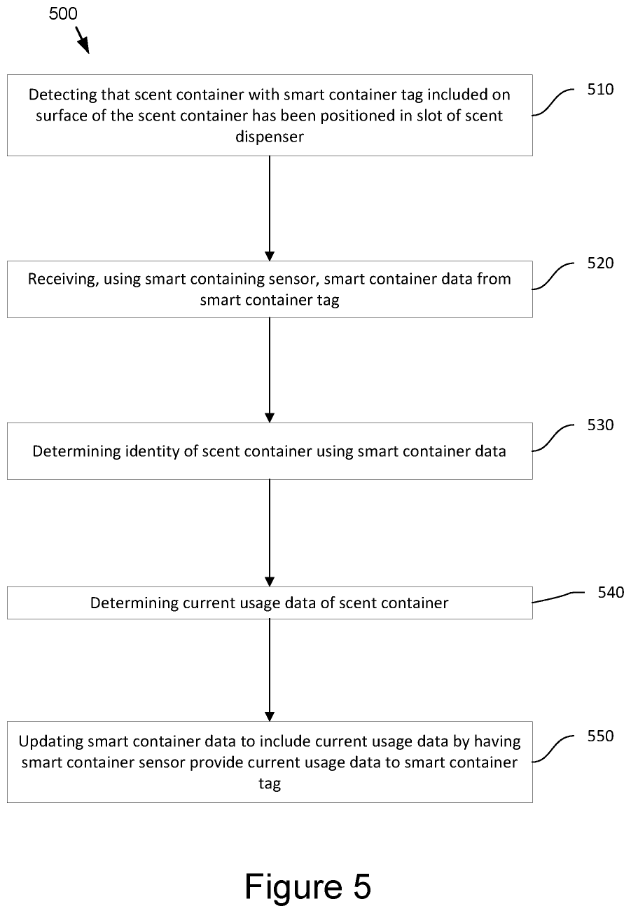

FIG. 5 is a flowchart of an example method for smart container detection. At step 510, a smart container sensor 199 detects that a scent container 250 with a smart container tag 197 included, such as on a surface of the scent container 250, has been positioned in a slot 280 of a scent dispenser 132. The smart container sensor 199 may detect that the scent container 250 is positioned in the first slot by detecting the smart container tag 197, such as by electrically coupling or wireless signals as described elsewhere herein. At step 520, the smart container sensor 199 may receive smart container data 183 from the smart container tag 197. At step 130, a controller 188 may receive the smart container data 183 from the smart container sensor 199 and determine an identity of the scent container 250 using the smart container data. In some implementations, the controller may determine the identity of the scent container 250 by identifying a unique identifier included in the smart container data 183 and may use the unique identifier to access additional smart container data 183 stored on the server 150 that includes the identity of the scent container 250 based on the unique identifier.

At step 540, the controller 188 may determine current usage data of the scent container 250, such as based on scent diffusion seatings, the heating element usage, etc. The current usage data may include data captured by the scent dispenser 132 that includes run-time for different scent containers 250, schedules of different scent containers 250, installations/refill of different scent containers 250, fill levels of different scent containers 250, etc. The current usage data may also include analytics data that is captured and/or generated by the dispenser management application 160 and stored on the server 150, which may then provide the current usage data to the controller 188 as needed. At step 550, the controller 188 may update the smart container data 183 to include the current usage data by having the smart container sensor 199 provide the current usage data to the smart container tag 197. In further implementations, the controller 188 may update the smart container data 183 to include the current usage data by providing the current usage data to the smart container data 183 stored on the server 150 and having the dispenser management application 160 update the stored smart container data 183. In some implementations, the dispenser management application 160 may retrieve and/or update the stored smart container data 183 based on the unique identifier detected on the smart container tag 197. Each of the smart container data 183 stored on the server 150 may be associated with different unique identifiers that are readable from smart container tags 197 on different scent containers 250.

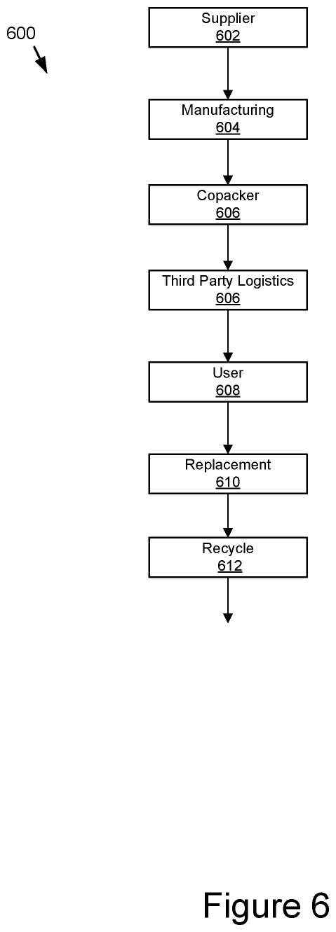

FIG. 6 is a block diagram of an example smart container tracking system. As described elsewhere herein, using a smart container tag 197 allows a scent container 250 to be tracked throughout the lifetime of the scent container 250. As the smart container 250 is manufactured through the end of use of the scent container 250, the smart container tag 197 can store various data related to the scent container 250. For example, as shown at 602, a supplier can prepare a smart container tag 197 for attaching to a scent container 250 during manufacturing. By preparing a smart container tag 197 that is unique for each scent container 250, the supplier can provide protection against fraudulent vials and ensure that the correct scent containers 250 are being used based on the unique identifier of each smart container tag 197. As each smart container tag 197 stores the data locally, such as on the circuit of the smart container tag 197, the data for the scent container 250 is tracked starting from the initial setup of the smart container tag 197.

At block 604, a manufacturer can build and assemble various scent containers 250 and affix them with a smart container tag 197, such as an NFC antenna. By affixing a unique smart container tag 197 to each scent container 250, the scent container functionality and compatibility with various devices can be easily tracked. Additionally, batch data and manufacturing environment information can be stored on the smart container tag 197 to assist in recalls or other situations, such as where a batch overperforms and the data can be accessed to identify any outliers that provide explanations.

At block 606, the copacker during manufacturing can fill each scent container 250 with fragrance and finalize the finished product. Using the smart container tag 197, each scent container 250 can connect unique product data directly to their unique smart container tag 197. This ensures additional data validity and/or provides ability to track expiration of fragrances by each scent container 250. For example, a batch of scent containers 250 can be packaged and then each scent container 250 includes their own packaged expiration data on each smart container tag 197 that automatically will provide updates and/or notifications when connected with a smart container sensor 199. At block 608, the third party logistics can package orders and send them out to customers. Using a smart container tag 197, each scent container 250 can be scanned and tracked by a separate smart container sensor 199 to update information and track various aspects of the scent containers 250. By tracking the smart container tag 197, the third party logistics can connect shipping location data to each unique smart container tag 197 and ensure data validity. By attaching the data to the smart container tag 197, that data is persistent with the scent container 250, not just the packaging or some other conventional tracking mechanism. This allows for tracking, under privacy considerations, to monitor customer behaviors, such as gifting, vial swapping, stealing of scent containers 250, retail conversion, etc.

At 608, a user 112 can install a scent container 250 and automatically have the smart container tag 197 read for relevant data. This allows a user 112 to order a product, unbox it, and install the vial without having to do any tracking or entering of serial numbers for registration, etc. The smart container tag 197 automatically connects and streamlines the user experience. Additionally, as the user 112 enjoys a scent experiences and plans for a lifetime of the scent container 250, the scent container 250 is able to monitor and track the relevant data related to usage settings and remaining scent, and store that locally on the scent container 250. This allows for that data to be persistent, even if the scent container 250 is moved to a different scent dispenser 132, regifted to a separate user 112, unplugged and unused for a period of time before being reinstalled, etc. This data remains with the scent container 250 and can quickly by used to provide updates to a user 112 as needed. The smart container tag 197 provides a connected time stamp when a scent container 250 is installed in a scent dispenser 132. It provides the ability for accurate remaining scent calculations using the various timestamps and automatically updates any scent container 250 changes, such as swapping a scent without the user 112 having to manually enter any information. Additionally, as the scent container 250 is used, all of the data is persistent and provides accurate messaging throughout the scent container 250 lifespan.

At 610, when a scent container 250 is removed, the time stamp of removal can be captured and stored on the smart container tag 197. This can be used to track when scent containers 250 are disposed of/refilled/replaced. A user does not have to do any manually entering of information when scent containers 250 are removed or switched out. Additionally, in some implementations, at 612, a recycling option can be used, where when a vial is empty or removed a supplier can automatically ship out a return box for a user to place the removed scent container 250 into. The scent container 250 is then tracked as it is returned and refilled for future use. In some implementations, the persistent data on the smart container tag 197 is preserved after being refilled for a full lifetime dataset of the scent container 250. While in further implementations, the smart container tag 197 is replaced when the scent container 250 is repurposed. By using a scent container 250 with a smart container tag 197 that automatically provides tracking data when installed, a user experience can be further streamlined and a more accurate data set of the use of the scent container 250 can be preserved for the scent container 250. In some implementations, the data set of the use of the scent container 250 based on the smart container tag 197 can be used with artificial intelligence and/or machine learning to generate additional analytics about the use of the scent container 250 and potential customer insights based on the generated additional analytics.

The foregoing description, for purpose of explanation, has been described with reference to various embodiments and examples. However, the illustrative discussions above are not intended to be exhaustive or to limit the invention to the precise forms disclosed. Many modifications and variations are possible in view of the above teachings. The various embodiments and examples were chosen and described in order to best explain the principles of the invention and its practical applications, to thereby enable others skilled in the art to utilize the innovative technology with various modifications as may be suited to the particular use contemplated. For instance, it should be understood that the technology described herein can be practiced without these specific details in some cases. Further, various systems, devices, and structures are shown in block diagram form in order to avoid obscuring the description. For instance, various implementations are described as having particular hardware, software, and user interfaces. However, the present disclosure applies to any type of computing device that can receive data and commands, and to any peripheral devices providing services.

In some instances, various implementations may be presented herein in terms of algorithms and symbolic representations of operations on data bits within a computer memory. An algorithm is here, and generally, conceived to be a self-consistent set of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout this disclosure, discussions utilizing terms such as “processing,” “computing,” “calculating,” “determining,” “displaying,” or the like, refer to the action and methods of a computer system that manipulates and transforms data represented as physical (electronic) quantities within the computer system's registers and memories into other data similarly represented as physical quantities within the computer system memories or registers or other such information storage, transmission or display devices.

A data processing system suitable for storing and/or executing program code, such as the computing system and/or devices discussed herein, may include at least one processor coupled directly or indirectly to memory elements through a system bus. The memory elements can include local memory employed during actual execution of the program code, bulk storage, and cache memories that provide temporary storage of at least some program code in order to reduce the number of times code must be retrieved from bulk storage during execution. Input or I/O devices can be coupled to the system either directly or through intervening I/O controllers. The data processing system may include an apparatus may be specially constructed for the required purposes, or it may comprise a general-purpose computer selectively activated or reconfigured by a computer program stored in the computer.

The foregoing description has been presented for the purposes of illustration and description. It is not intended to be exhaustive or to limit the specification to the precise form disclosed. Many modifications and variations are possible in light of the above teaching. It is intended that the scope of the disclosure be limited not by this detailed description, but rather by the claims of this application. As will be understood by those familiar with the art, the specification may be embodied in other specific forms without departing from the spirit or essential characteristics thereof. Likewise, the particular naming and division of the modules, routines, features, attributes, methodologies and other aspects may not be mandatory or significant, and the mechanisms that implement the specification or its features may have different names, divisions, and/or formats.

Furthermore, the modules, routines, features, attributes, methodologies and other aspects of the disclosure can be implemented as software, hardware, firmware, or any combination of the foregoing. The technology can also take the form of a computer program product accessible from a computer-usable or computer-readable medium providing program code for use by or in connection with a computer or any instruction execution system. Wherever a component, an example of which is a module or engine, of the specification is implemented as software, the component can be implemented as a standalone program, as part of a larger program, as a plurality of separate programs, as a statically or dynamically linked library, as a kernel loadable module, as firmware, as resident software, as microcode, as a device driver, and/or in every and any other way known now or in the future. Additionally, the disclosure is in no way limited to implementation in any specific programming language, or for any specific operating system or environment. Accordingly, the disclosure is intended to be illustrative, but not limiting, of the scope of the subject matter set forth in the following claims.

What is claimed is:

1. A scent dispensing apparatus, comprising:
a scent container retaining apparatus configured to receive and securely retain a scent container, wherein the scent container includes a smart vial tag;
a smart vial sensor directionally positioned to have a direct line of sight with the smart vial tag to read the smart vial tag when the scent container is securely retained in the scent retaining apparatus; and
a fragrance management application that receives an identity of the smart vial tag from the smart vial sensor and generates a unique container profile for the scent container.

2. The scent dispensing apparatus of claim 1, wherein the smart vial sensor is an optical sensor and the smart vial tag is a QR code detectable by the smart vial sensor.

3. The scent dispensing apparatus of claim 1, wherein the smart vial sensor is a radio receiver and the smart vial tag is an RFID tag that can be read by the radio receiver.

4. The scent dispensing apparatus of claim 3, wherein the radio receiver is a directional antenna positioned within a threshold distance of the RFID tag when the scent container retaining apparatus retains the scent container.

5. The scent dispensing apparatus of claim 1, wherein the unique container profile includes one or more of an identity of a scent solution, settings of a scent solution, states of the scent container, a fill-level of the scent container, and a location of the scent container.

6. The scent dispensing apparatus of claim 1, wherein the scent container includes a scent solution that can be emitted from the scent container.

7. The scent dispensing apparatus of claim 1, wherein the smart vial tag further includes smart container data that can be read from the smart vial tag.

8. The scent dispensing apparatus of claim 1, wherein the smart vial tag is located on a bottom surface of the scent container.

9. The scent dispensing apparatus of claim 1, wherein the unique container profile includes historical data of the scent container.

10. A method of using a scent dispensing apparatus, comprising:
detecting, using a smart container sensor of a scent dispensing apparatus, that a scent container with a smart container tag positioned on a surface of the scent container has been positioned in a slot of the scent dispensing apparatus;
receiving, using the smart container sensor, smart container data from the smart container tag;
determining, using a processor of the scent dispensing apparatus, an identity of the scent container using the smart container data;
determining, using the processor of the scent dispensing apparatus, current usage data of the scent container; and
updating, using the processor of the scent dispensing apparatus, the smart container data to include the current usage data by having the smart container sensor provide current usage data to the smart container tag of the smart container.

11. The method of claim 10, wherein the smart container sensor is a radio sensor and the smart container tag is a RFID tag that can be read by the radio sensor when the RFID tag is within a threshold distance of the radio sensor.

12. The method of claim 11, wherein the RFID tag is positioned on a bottom surface of the scent container and the radio sensor is positioned below where the scent container is positioned in the scent dispensing apparatus.

13. The method of claim 11, wherein the radio sensor is a directional antenna and wherein when the scent container is positioned in the scent dispensing apparatus, the radio sensor has a direct line of sight with the RFID tag.

14. The method of claim 10, wherein the smart container data includes a unique identifier that represents the identity of the scent container.

15. The method of claim 10, wherein the smart container sensor is an optical sensor and the smart container tag is a QR code that is detectable by the optical sensor.

16. The method of claim 10, wherein the smart container sensor includes electrical sensor contacts configured to engage with electrical tag contacts on the smart container tag.

17. The method of claim 10, wherein the scent container includes a scent solution that can be emitted from the scent container.

18. The method of claim 10, wherein the current usage data includes one or more of an identity of a scent solution, settings of a scent solution, states of the scent container, a fill-level of the scent container, and a location of the scent container.

19. The method of claim 10, wherein updating the smart container data to include the current usage data further comprises:
tracking the smart container tag during a manufacturing process to include first tracking data;
tracking the smart container tag during a shipping process to include second tracking data; and
updating the smart container data to include the first tracking data and the second tracking data.

20. A smart container tag tracking method comprising:
installing a smart container tag with a unique identifier on a scent container during manufacturing;
tracking the smart container tag during a manufacturing process;
tracking the smart container tag during a shipping process;
automatically connecting the smart container tag to a scent dispenser using a smart container sensor;
updating a scent container lifetime based on the tracking of the smart container tag;
applying a timestamp at removal of the scent container; and
updating the smart container tag with the timestamp.
